(19) United States
(12) Patent Application Publication
(10) Pub. No.: US 2025/0325406 A1
(43) Pub. Date: Oct. 23, 2025

(54) BANDAGE DEVICE

(71) Applicant: Tanner Seehawsen, Mesa, AZ (US)

(72) Inventor: Tanner Seehawsen, Mesa, AZ (US)

(21) Appl. No.: 19/184,614

(22) Filed: Apr. 21, 2025

Related U.S. Application Data
(60) Provisional application No. 63/636,133, filed on Apr. 19, 2024.

(51) Int. Cl. A61F 13/02; A61F 13/00

(52) U.S. Cl. CPC A61F 13/0273; A61F 2013/00153; A61F 2013/00314

(57) ABSTRACT

A bandage device is disclosed that provides a specialized fingertip bandage. The bandage device is comprised of a regular bandage having a T-shaped style. The body component comprises the main bandage shape that is adhesive on the rear face side. A wing component is also integrated into the body component to help secure the body component on the fingertip but can be easily separated from the device with scissors or along a perforated edge. Thus, each bandage device functions as a suitable way of putting a bandage on a fingertip. The device can also be decorated for pleasure, as well.

### CROSS-REFERENCE TO RELATED APPLICATION

The present application claims priority to, and the benefit of, U.S. Provisional Application No. 63/636,133, which was filed on Apr. 19, 2024, and is incorporated herein by reference in its entirety.

### FIELD OF THE INVENTION

The present invention relates generally to the field of bandage devices. More specifically, the present invention relates to a specialized fingertip bandage. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in bandage devices. Generally, injuries to the area on and around the fingertip can be painful and frustrating to deal with. Applying standard bandages to the fingertip can be difficult, and often the bandage does not properly wrap around the finger and remain secure. Accordingly, people may be looking for safer, more secure, and more unique ways to apply bandages to your fingertips.

Further, the use of bandages to cover a fingertip is well-known in the prior art. However, it would be desirable to include an easier way for the bandage to cover the fingertip and stay secure on the fingertip. For example, it would be desirable to provide an improved bandage that could stay on the fingertip for longer and prevent the wound from becoming infected. Constantly, reapplying bandages to a fingertip wound can be frustrating and annoying. Accordingly, standard bandages do not properly wrap around fingertip wounds.

Accordingly, there is a demand for an improved bandage device that can be applied to fingertip wounds specifically. More particularly, there is a demand for a specialized fingertip bandage that can properly wrap around a fingertip wound to protect scrapes, cuts, lacerations, or other minor wounds.

### SUMMARY OF THE INVENTION

There exists a long-felt need in the art for a bandage device that provides users with a bandage that can be specifically applied to fingertip wounds and injuries. There is also a long-felt need in the art for a bandage device that features a T-shaped structure in which the central bandage is surrounded by a pair of adhesive wings such that users can wrap the wings around the fingertip to secure the bandage. Further, there is a long-felt need in the art for a bandage device that offers case of application for bandages being applied to the fingertip area. Moreover, there is a long-felt need in the art for a device that eliminates the need to constantly replace standard bandages or leave a wound uncovered due to frustrating bandage application. Further, there is a long-felt need in the art for a bandage device that allows users to cut off the adhesive wings and utilize the device as a standard bandage. Finally, there is a long-felt need in the art for a bandage device that can be decorated in any way desirable for the user.

The subject matter disclosed and claimed herein, in one embodiment thereof, comprises a bandage device. The device is a specialized fingertip bandage. The bandage device is comprised of a regular bandage having a T-shaped style. The body component comprises the main bandage shape that is adhesive on one side. A wing component is also integrated into the body component but can be easily separated from the device with scissors. Thus, each bandage device functions as a suitable way of putting a bandage on a fingertip. The device can also be decorated for pleasure, as well.

In this manner, the bandage device of the present invention accomplishes all of the foregoing objectives and provides users with a device that protects fingertip wounds and injuries. The device is an adhesive bandage with wings that can properly wrap around the fingertip. The device can be manufactured of traditional bandage material and the wings can be detached if necessary.

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a bandage device. The device is a specialized fingertip bandage. The bandage device comprises a body component that is configured in a regular bandage shape. The body component also comprises a wing component which gives it a T-style shape. Thus, each bandage device functions as a suitable way of protecting a fingertip wound, through wrapping the bandage device around the wound.

In one embodiment, the bandage device comprises a body component and a wing component. The body component is adapted to wrap completely around the tip of a finger to provide maximum protection. The bandage device is configured in a T-shape.

In one embodiment, the bandage device comprises a body component that is shaped like a regular bandage. The body component is also made of the same material as other previously known bandages and functions similarly as well. The body component also comprises a wing component which is comprised of the same material as the body component and attached to one end of the body component to form a T-shaped structure. Both the body component and the wing component have an adhesive on one face in order to allow the bandage to stick to skin.

In one embodiment, the wing component is utilized to completely wrap around the tip of the finger and allow for maximum coverage for small fingertip wounds. The wing component is made of a cotton material to make it lightweight, breathable, and absorbent. The adhesive on one side of the bandage is typically made of an acrylic polymer and covered by an adhesive release sheet which must be taken off before use.

In one embodiment, the body component is configured in the shape of a regular bandage, including the sanitary pad in the center of the bandage which touches against the wound. This absorbent pad is made of cotton as well and could be dipped in antiseptic solution for prevention against infections. The body component also comprises adhesive on the same face as the wing component and made from the same material along with an adhesive release sheet covering the adhesive as well.

In one embodiment, the wing component can be detached from the body component easily by cutting the wings off with scissors, although the scissors are not included in the invention. In another embodiment, the wing component can be detached from the body component by tearing at a perforated line that is present between the wing component and the body component for this purpose.

In one embodiment, the bandage device can be any suitable color or design suitable in the art for extra appeal. The cloth outside of the bandage device can also be manufactured of an adhesive bandage film instead of cloth to allow the bandage device to be waterproof.

In use, the bandage device is assembled by first removing the adhesive release sheet from the back face of the device. When the bandage device is properly positioned, the wing component is wrapped around the sides of the fingertip and the adhesive is used to stick to the skin. The body component is then secured to the skin via adhesive as well so that the absorbent pad is against the wound. The wing component allows the bandage device to be removably secured to the fingertip in use, as needed. Thus, a scrape, cut, laceration, or other minor wound, inside the body component remains protected from infection or other pain.

In one embodiment, the bandage device can be utilized with any finger on the user's hand or even with a toe on the user's foot.

In yet another embodiment, the bandage device comprises a plurality of indicia.

In yet another embodiment, a method of protecting a fingertip wound is disclosed. The method includes the steps of providing a bandage device comprising a body component and wing component that are a T-shaped structure with adhesive and an adhesive sheet. The method also comprises removing the adhesive sheet from the back face of the body and wing component. Further, the method comprises securing the device onto the fingertip by wrapping the wing component around the finger. Finally, the method comprises sticking the bandage device to the skin with the adhesive on the back face of the wing component and body component.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of a bandage device of a present invention showing how the device is applied in accordance with an embodiment;

FIG. 2 illustrates a perspective view of one embodiment of the bandage device of the present invention showing how the adhesive sheet protects the adhesive on the device in accordance with an embodiment;

FIG. 3 illustrates a perspective view of one embodiment of the bandage device of the present invention showing the device in use in accordance with an embodiment;

FIG. 4 illustrates a perspective view of one embodiment of the bandage device of the present invention showing the device in use in accordance with an embodiment;

FIG. 5 illustrates a perspective view of one embodiment of the bandage device of the present invention showing the sort of wound that the device would be covering in accordance with an embodiment; and

FIG. 6 illustrates a flowchart showing the method of protecting a fingertip wound in accordance with an embodiment.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a bandage device that provides users with a bandage that can be specifically applied to fingertip wounds and injuries. There is also a long-felt need in the art for a bandage device that features a T-shaped structure in which the central bandage is surrounded by a pair of adhesive wings such that users can wrap the wings around the fingertip to secure the bandage. Further, there is a long-felt need in the art for a bandage device that offers case of application for bandages being applied to the fingertip arca. Moreover, there is a long-felt need in the art for a bandage device that eliminates the need to constantly replace standard bandages or leave a wound uncovered due to frustration with bandage applications. Further, there is a long-felt need in the art for a bandage device that allows users to cut off the adhesive wings and utilize the device as a standard bandage, if desired. Finally, there is a long-felt need in the art for a bandage device that can be decorated in any way that is desirable for the user.

The present invention, in one exemplary embodiment, is a novel bandage device. The device is a specialized fingertip bandage. The bandage device comprises a body component that is configured in the shape of a traditional bandage with the same measurements. The body component would be the same cloth material that usually makes up a traditional bandage as well. The body component also comprises a wing component which is made of the same cloth material as the body component. Thus, each bandage device functions as a suitable way of protecting a wound on a fingertip, through wrapping the wing component around the fingertip and the body component covering the wound. The present invention also includes a novel method of protecting a fingertip wound. The method includes the steps of providing a bandage device comprising a body component and wing component that are a T-shaped structure with adhesive and an adhesive sheet. The method also comprises removing the adhesive sheet from the back face of the body and wing component. Further, the method comprises securing the device onto the fingertip by wrapping the wing component around the finger. Finally, the method comprises sticking the bandage device to the skin with the adhesive on the back face of the wing component and body component.

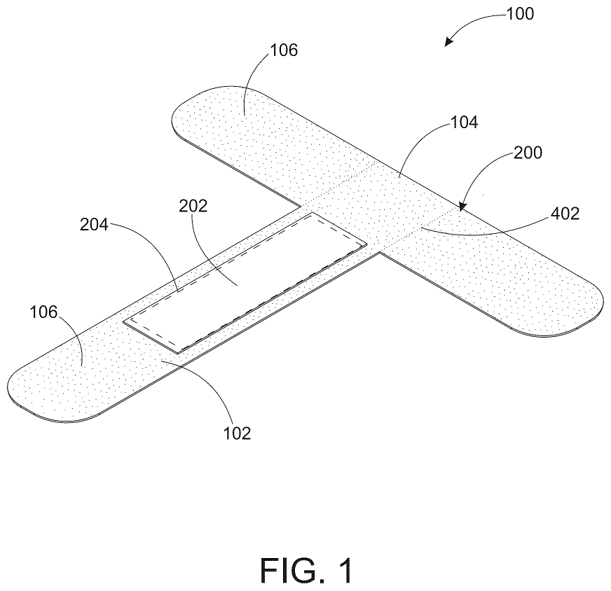

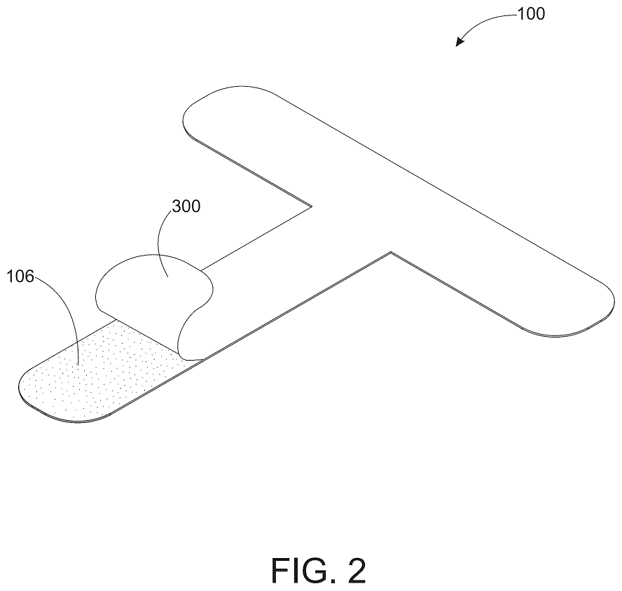

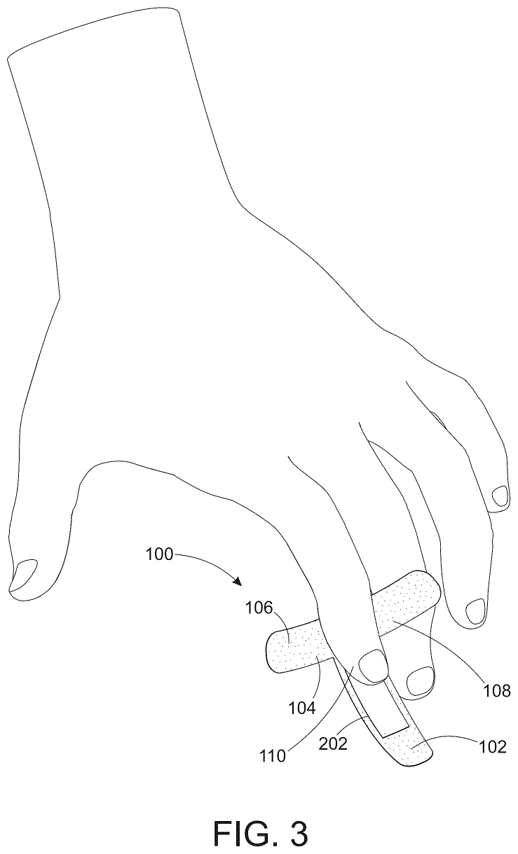

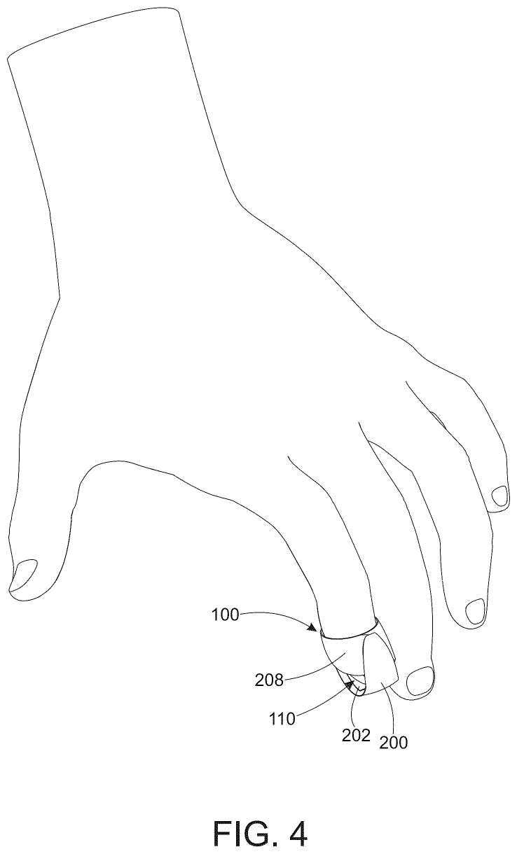

Referring initially to the drawings, FIGS. 1-4 illustrate views of one embodiment of the bandage device 100 of the present invention. In the present embodiment, the bandage device 100 is an improved bandage device 100 that provides a user with a specialized fingertip 110 bandage. Further, the device 100 is comprised of a regular bandage with a T-shaped structure. Specifically, the bandage device 100 comprises a body component 102 with a wing component 104 and adhesives 106 for securing the device 100 to the user's skin 108. Thus, each bandage device 100 functions as a suitable way of protecting a fingertip 110 wound 112 from infection and pain.

Generally, the bandage device 100 comprises a body component 102, a wing component 104, and adhesives 106. The body component 102 is adapted to wrap around the fingertip 110 in order to keep the wound 112 safe. Further, the wing component 104 allows extra security around the fingertip 110 and a more comfortable means of securing the bandage device 100.

The bandage device 100 comprises a body component 102 that is in the shape of a regular bandage. Further, the body component 102 comprises an adhesive 106 on the back face 200 of the body component 102, wherein the adhesive 106 is applied to the entire back face 200. This adhesive 106 allows for the body component 102 to stick to the skin 108 and allows the body component 102 to remain secure on the fingertip 110. Also on the back face 200 of the body component 102 is an absorbent pad 202 which is covered in antiseptic 204 to protect the wound 112 from infection. The absorbent pad 202 is placed directly on top of the wound 112 and is surrounded by the adhesives 106.

Additionally, a wing component 104 is utilized to add additional security and comfort to the wound 112. The wing component 104 is made of the same material 206 as an original bandage and also contains the same adhesives 106 on the back face 208 of the wing component 104, as well, wherein the adhesive 106 is applied to the entire back face 208 of the wing component 104. The wing component 104 allows the whole device 100 to be structured in a T-shape.

The body component 102 comprises an adhesive release sheet 300 that covers the adhesives 106 on the back face 200 of the body component 102 as well as the back face 208 of the wing component 104. The adhesive release sheet 300 is then removed from the device 100 before the adhesives 106 can be stuck to the skin 108.

The wing component 104 can be removed from the device 100 if desirable so that the device 100 is more like a traditional bandage. The wing component 104 can be removed from the rest of the device 100 with scissors (not shown) cutting the device 100 into whatever shape is conducive to the wound 112. In another embodiment, the wing component 104 is attached to the body component 102 by perforated edges 402 which allows the wing component 104 to be easily torn from the body component 102 when not needed.

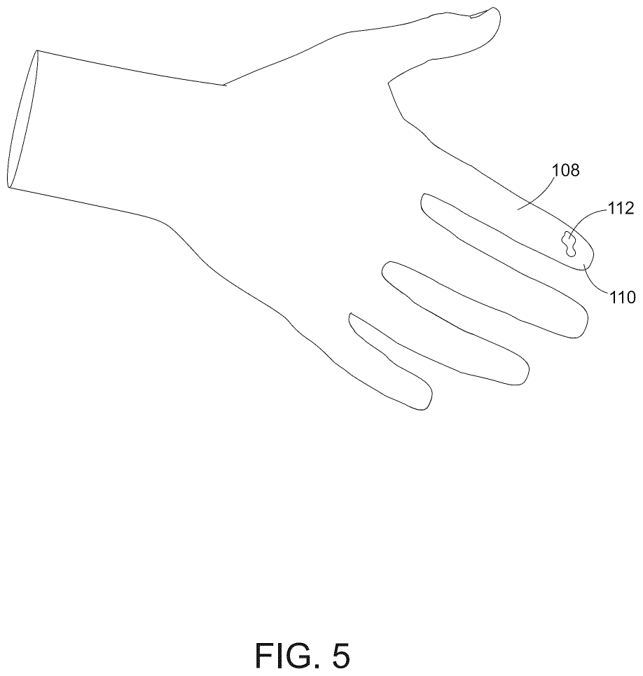

As shown in FIG. 5, the bandage device 100 can be utilized with any suitable scrape, cut, laceration, wound, etc., 112, as is known in the art, and even with toes as well as fingertips (not shown).

In one embodiment, the bandage device 100 comprises a plurality of indicia not shown. The body component 102 of the device 100 may include advertising, a trademark, or other letters, designs, or characters, printed, painted, stamped, or integrated into the body component 102, or any other indicia as is known in the art. Specifically, any suitable indicia as is known in the art can be included, such as, but not limited to, patterns, logos, emblems, images, symbols, designs, letters, words, characters, animals, advertisements, brands, etc., that may or may not be bandage, wound, or brand related.

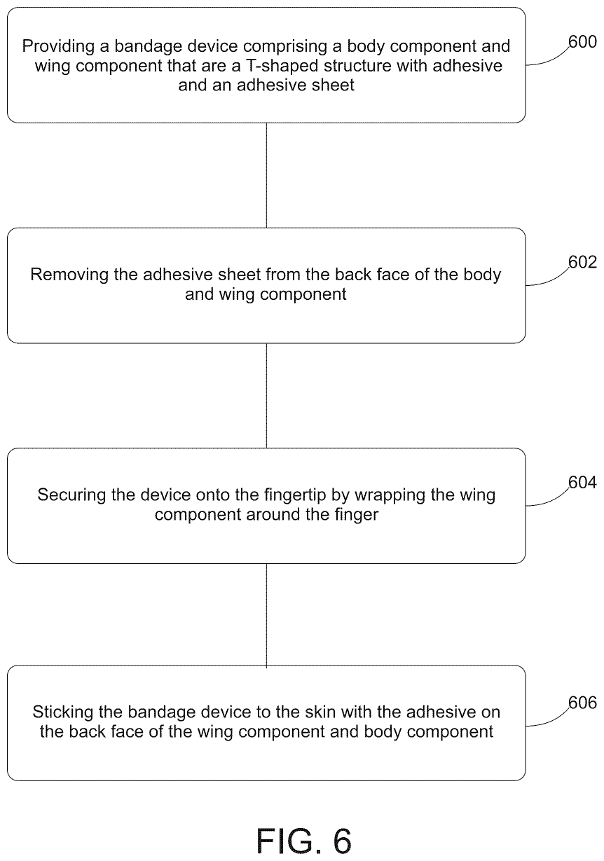

FIG. 6 illustrates a flowchart of the method of protecting a fingertip wound. The method includes the steps of at 600, providing a bandage device comprising a body component and wing component that are a T-shaped structure with adhesive and an adhesive sheet. The method also comprises at 602 removing the adhesive sheet from the back face of the body and wing component. Further, the method comprises at 604 securing the device onto the fingertip by wrapping the wing component around the finger. Finally, the method comprises at 606 sticking the bandage device to the skin with the adhesive on the back face of the wing component and body component.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “bandage device”, “fingertip device”, “fingertip bandage device”, and “device” are interchangeable and refer to the bandage device 100 of the present invention.

Notwithstanding the foregoing, the bandage device 100 of the present invention can be of any suitable size and configuration as is known in the art without affecting the overall concept of the invention, provided that it accomplishes the above-stated objectives. One of ordinary skill in the art will appreciate that the bandage device 100 as shown in FIGS. 1-6 is for illustrative purposes only, and that many other sizes and shapes of the bandage device 100 are well within the scope of the present disclosure. Although the dimensions of the bandage device 100 are important design parameters for user convenience, the bandage device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bandage device that provides a user with a specialized fingertip bandage for protecting a fingertip wound, the bandage device comprising:
a body component; and
a wing component;
wherein the body component and the wing component are adapted to secure onto a fingertip and protects a wound on the fingertip; and
further wherein the body component and wing component are secured to the fingertip via adhesives.

2. The bandage device of claim 1, wherein the body component is a cloth structure configured in a traditional bandage shape.

3. The bandage device of claim 2, wherein the body component comprises a back face which is coated in adhesive.

4. The bandage device of claim 3, wherein a rear body component contains an absorbent pad where the bandage device connects with the fingertip wound.

5. The bandage device of claim 4, wherein the wing component comprises a back face coated in adhesive as well.

6. The bandage device of claim 5, wherein the wing component can be detached from the body component with scissors.

7. The bandage device of claim 6, wherein the wing component could also be detached from the body component by tearing at a perforated edge which is situated between the wing component and the body component.

8. The bandage device of claim 7, wherein adhesives on the back face of the body component and wing component are covered by an adhesive release sheet which is removed in order to use the bandage device.

9. The bandage device of claim 8, wherein the body component and wing component could be various colors or designs.

10. The bandage device of claim 9, wherein the body component and wing component form a T-shaped structure.

11. The bandage device of claim 10, wherein the bandage device can be utilized with a toe.

12. The bandage device of claim 11 further comprising a plurality of indicia.

13. A bandage device that provides a user with a specialized fingertip bandage, the bandage device comprising:
a body component comprising a rear face coated in adhesives;
a wing component comprising a rear face coated in adhesives; and
an adhesive release sheet;
wherein the body component is a cloth structure configured in a traditional bandage shape;
wherein the body component has an absorbent pad which covers a wound;
wherein the wing component and body component are attached to form a T-shaped structure;
wherein the wing component wraps around a fingertip to secure the body component in place;
wherein the adhesives are used to secure the bandage device onto a user's skin; and
further wherein the body component protects the fingertip wound from infection or pain.

14. The bandage device of claim 13, wherein material of both the body component and the wing component can be elastic to allow the bandage device to be waterproof.

15. The bandage device of claim 13, wherein the wound comprises a scrape, cut, laceration, or other minor wound.

16. The bandage device of claim 13, wherein the adhesives are made up of an acrylic polymer and covered by the adhesive release sheet.

17. The bandage device of claim 13 further comprising a plurality of indicia.

18. The bandage device of claim 13, wherein the absorbent pad is dipped in antiseptic to further prevent infection.

19. The bandage device of claim 13, wherein the wing component can be removed from the body component through cutting with scissors or ripping along a perforated edge.

20. A method of protecting a fingertip wound, the method comprising the following steps:
providing a bandage device comprising a body component and wing component that are a T-shaped structure with adhesive and an adhesive sheet;
removing the adhesive sheet from the back face of the body and wing component;
securing the device onto the fingertip by wrapping the wing component around the finger; and
sticking the bandage device to the skin with the adhesive on the back face of the wing component and body component.
